(19) United States
(12) Patent Application Publication
(10) Pub. No.: US 2022/0412972 A1
(43) Pub. Date: Dec. 29, 2022

(54) DIAGNOSIS METHOD USING PLASMON PHENOMENON, DIAGNOSTIC KIT AND MANUFACTURING METHOD OF DIAGNOSTIC KIT

(71) Applicant: KYUNGPOOK NATIONAL UNIVERSITY INDUSTRY-ACADEMIC COOPERATION FOUNDATION, Buk-gu (KR)

(72) Inventors: Sung Wook NAM, Dalseo-gu (KR); Moon Kyu KWAK, Buk-gu (KR); Hye Jin LEE, Buk-gu (KR)

(21) Appl. No.: 17/825,935

(22) Filed: May 26, 2022

(30) Foreign Application Priority Data
May 26, 2021 (KR) 10-2021-0067850

(51) Int. Cl. G01N 33/543; G01N 33/553

(52) U.S. Cl. CPC G01N 33/54346; G01N 33/553; B82Y 5/00

(57) ABSTRACT

Disclosed is a method for diagnosing a target material by using a first substrate printed with a first nanoparticle. A second nanoparticle, which is bonded to a compound to be bound to the target material, is positioned at a distance adjacent to the first substrate.

### TECHNICAL FIELD

Embodiments of the inventive concept described herein relate to a diagnosis method and a diagnosis kit using plasmon phenomenon, and a method for manufacturing a diagnosis kit.

### BACKGROUND ART

Plasmon refers to a mode generated by collective oscillation of metal atoms and free electrons present around the atoms. A localized surface plasmon resistance (LSPR) sensor refers to a sensing technique for measuring a fine change in refractive index, which results from a change in the surrounding environment of a metal nanostructure, through a change in absorbance.

Conventionally, a sensor using a plasmon phenomenon been disclosed. However, there is a limitation in the concentration of a protein to be detected.

### CONTENTS OF THE INVENTION

### Problems to be Solved

Embodiments of the inventive concept provide a technology for detecting a protein-based biomarker biomaterial with higher sensitivity, based on a plasma sensor having a nanoparticle-nanopattern coupled to each other, and suggest a method for utilizing a diagnostic device and a diagnostic kit.

The objects of the inventive concept are not limited to the above, but other effects, which are not mentioned, will be apparently understood to those skilled in the art. Other problems which are not mentioned will be clearly understood from the following description to those skilled in the art.

### Solutions to the Problems

According to an embodiment, there is disclosed a method for diagnosing a target material by using a first substrate printed with a first nanoparticle.

According to an embodiment, the method may include positioning a second nanoparticle, which is bonded to a compound to be bound to the target material, at a distance adjacent to the first substrate.

According to an embodiment, a compound to be coupled to the target material may be bonded to the first nanoparticle.

According to an embodiment, the method may include applying the target material.

According to an embodiment, the method may include detecting the target material through a plasmon phenomenon of the first nanoparticle and the second nanoparticle.

According to an example, a shape of the second nanoparticle may be one of a cube, a rectangular parallelepiped, a sphere, and a cylinder.

According to an embodiment, the first nanoparticle and the second nanoparticle may be one of metal causing a plasmon phenomenon.

According to an embodiment, the adjacent distance may be in the range of 30 nm to 200 nm.

According to an embodiment, there is disclosed a diagnostic kit including a first substrate printed with a first nanoparticle to diagnose the target material.

According to an embodiment, the diagnosis kit may further include a second nanoparticle be coupled to the first nanoparticle.

According to an embodiment, a compound to be bound to the target material may be bonded to the first nanoparticle and the second nanoparticle.

According to an embodiment, the second nanoparticle may be provided at a distance adjacent to the first nanoparticle.

According to another embodiment, there is a method for manufacturing a diagnosis kit to diagnose a target material.

According to an embodiment, the method includes printing a first nanoparticle on a first substrate, and attaching a diagnosis kit printed with the first nanoparticle into a plate.

According to an embodiment, the method may include bonding a compound, which is to be bound to the target material, to the first nanoparticle.

According to an embodiment, the method may include positioning a second nanoparticle, which is bonded to a compound to be bound to the target material, into the plate.

### Advantages of the Invention

According to the inventive concept, the nanostripe structure platform may be mass-produced by utilizing a nanoimprint technology.

According to the inventive concept, colloidal-based nanoparticles used for a sample test may be mass-produced through a nanoparticle synthesis technology.

According to the inventive concept, a kit for diagnosing a disease may be configured based on a nanostripe-based plasmon material platform and nanoparticle-based sample test reagent.

According to an embodiment of the inventive concept, a protein-based biomarker can be detected, and thus a kit or a sensor for diagnosing a disease, such as a cancer, diabetes, and degenerative brain a disease, may be provided.

According to an embodiment, when compared to a conventional technology, even a significantly smaller amount of protein may be sensed.

The effects of the inventive concept are not limited to the above effects. Any other effects not mentioned herein will be clearly understood from the following description by those skilled in the art to which the inventive concept pertains.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a view illustrating that a first nanoparticle is printed on a first substrate, according to the inventive concept;

FIGS. 2A to 2D are views illustrating a printing scheme of FIG. 1, in more detail;

FIGS. 3A to 3C are views illustrating a method for fabricating a second nanoparticle, according to the inventive concept;

FIGS. 4A to 4E are views illustrating that the second nanoparticle is prepared, according to the scheme of FIGS. 3A to 3C;

FIGS. 5A to 5C are views illustrating a diagnosis method and a plasmon phenomenon, according to the inventive concept;

FIGS. 6A to 6D are views illustrating results obtained by analyzing results of a nanoparticle having a surface bio-functionalized with an antibody through inductively coupled plasma;

FIGS. 7A to 7I are views illustrating analysis results through a photoelectron spectroscopy, based on whether a compound is bonded to a first substrate printed with a nanoparticle;

FIG. 8A to 8D illustrates results determined through a spectroscope after an antibody is fixed;

FIGS. 9A to 9F are views illustrating a method for manufacturing a diagnosis kit and a diagnosis method, according to an embodiment of the inventive concept;

FIGS. 10A to 10F are views illustrating that a target material is detected, according to an embodiment; and

FIG. 11 illustrates a table showing the comparison between control signals in a diagnosis kit, according to an embodiment of the inventive concept.

### DETAILED DESCRIPTION

Various embodiments of the inventive concept will be described more fully with reference to the accompanying drawings to such an extent as to be easily embodied by one skilled in the art. However, the inventive concept may be embodied in many different forms and should not be construed as limited to the embodiments set forth herein. In addition, in the following description of the inventive concept, a detailed description of well-known art or functions will be ruled out in order not to unnecessarily obscure the gist of the inventive concept. In addition, parts performing similar functions and similar operations will be assigned with the same reference numerals throughout the drawings.

When a certain part “includes” a certain component, the certain part does not exclude other components, but may further include other components if there is a specific opposite description. In detail, It will be further understood that the terms “comprises,” “comprising,” “includes,” or “including,” or “having” specify the presence of stated features, numbers, steps, operations, components, parts, or the combination thereof, but do not preclude the presence or addition of one or more other features, numbers, steps, operations, components, components, and/or the combination thereof.

The singular forms are intended to include the plural forms unless the context clearly indicates otherwise. In addition, the shapes and the sizes of elements in accompanying drawings will be exaggerated for more apparent description.

Although the terms first, second, etc. may be used herein to describe various components, these components should not be limited by these terms These terms are used to distinguish one component from another component. For example, a first component discussed below could be termed a second component without departing from the technical scope of the inventive concept. Similarly, the second component could be termed the first component.

The inventive concept suggests a diagnosis kit and a diagnosis method, capable of sensing an extreme small amount of sample, which may be not detected in a single structure, by coupling a first nanoparticle 100, which is printed on a first substrate 10, to a second nanoparticle 200 adjacent to the first nanoparticle 100 in structure, thereby enhancing the sensitivity of a sensor. According to the inventive concept, a plasmon is amplified based on a physical property. According to an embodiment of the inventive concept, a plasmon signal is amplified by coupling at least two plasmon nanostructures, which include a plasmon material, such as gold (Au), silver (Ag), and platinum (Pt), to each other, thereby enhancing the sensitivity of the sensor. According to an embodiment of the inventive concept, the form in which compounds 110 and 210 are bound to each other through a target material CT′, in which the compounds 110 and 210 are bound to a nanostripe platform structure based on nanoimprint (that is, the first nanoparticle 100 formed on the first substrate 10) and a colloidal-based nanoparticle structure (that is, the second nanoparticle 200), respectively. The first nanoparticle 100 may have a nanostructure or a nanopattern formed through a top-down scheme such as a deposition scheme, and may have a stripe structure. The second micro-lenses 151 may have a nanostructure or a nanopattern formed through a bottom-up scheme, and may be a colloidal-based nanoparticle formed through chemical synthesis.

According to the inventive concept, the first nanoparticle 100 and the second nanoparticle 200 may be provided in the binding form of antibody 110-antigen ‘T’-antibody 210. In this case, the antibody may be a compound, and the antigen may be a target material T, but the inventive concept is not limited thereto.

The inventive concept suggests a scheme for detecting an antibody in a nanomole level or a picomole level by amplifying a plasmon phenomenon, as a sandwich structure of an antibody-antigen-antibody form is made by depositing a metal material on the first substrate 10 through a nanoimprint scheme such that the metal material have a nanostripe form, thereby deriving a plasmon, and then synthesizing another metal nanoparticle. Hereinafter, a diagnosis method, a diagnosis kit, and a method for manufacturing the diagnosis method according to the inventive concept will be described in detail with reference to accompanying drawings.

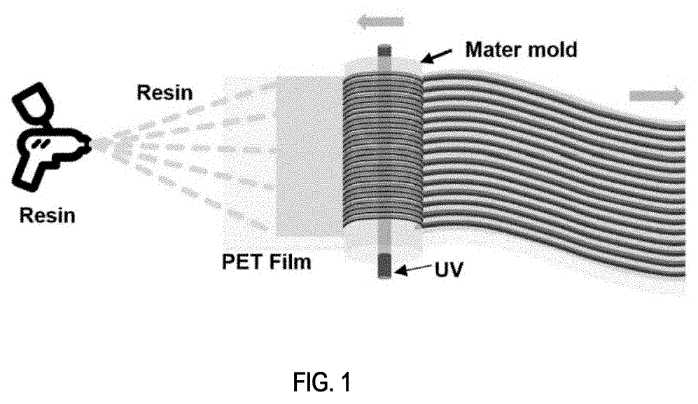

FIG. 1 is a view illustrating that a first nanoparticle 100 is printed on a first substrate 10, according to the inventive concept.

Referring to FIG. 1, a chip having a nanostructure may be mass-produced through a roll-to-roll nanoimprint fabrication technology, and metal is coated to the resultant structure through a thermal deposition process, thereby manufacturing a plasmon sensor. In this case, the metal may include gold (Au), silver (Ag), and platinum (Pt). However, the metal for the deposition is not limited thereto, and various metals may be employed, as long as the metals cause a plasmon phenomenon. In this case, the nanopattern formed on the first substrate 10 may include a nanostripe structure, a nanodot structure, and a nanosquare structure. However, the nanopattern is not limited thereto.

According to the inventive concept, when the metal is thermally deposited to the nanostructure pattern formed on the first substrate 10, the size and the shape of the nanostructure may be adjusted by inclining a deposition stage, such that the coated metal has a polymorphism structure.

FIG. 1 is a schematic view of fabricating a nanostripe through a roll-to-roll nanoimprint technology, after coating a resin on a flexible substrate, such as a PET film, by using an air brush. According to the inventive concept, a nanostructure may be fabricated on the flexible substrate in a larger amount within a shorter period of time, through the roll-to-roll nanoimprint technology. In addition, a nanopattern engraved into a master mold may be transferred to the flexible substrate, and may be mass-produced on the flexible substrate by using a UV-curable resin. According to an embodiment, the first substrate 10 may be the flexible substrate. According to an embodiment, the first substrate 10 may include any one of a carbon (C)-based polymer, such as polyethylene terephthalate (PET), polyimide (PI), polyethylene (PE), polyvinyl chloride (PVC), polypropylene (PP), polystyrene (PS), poly(tetrafluoroethylene) (PTFE, Teflon), or poly(acrylonitrile), poly(methyl methacrylate) (PMMA, Plexiglas) or a silicon (Si)-based polymer such as polydimethylsiloxane (PDMS). According to an embodiment, the first nanoparticle 100 printed on the first substrate 10 may be provided in the form of the nanopattern. In this case, the nanopattern may include a nanostripe structure, a nanodot structure, and a nanosquare structure. According to an embodiment of the inventive concept, the PET film having a metal nanopattern structure is translucent, so light is transmitted through the PET film. Accordingly, the change in absorbance may be observed.

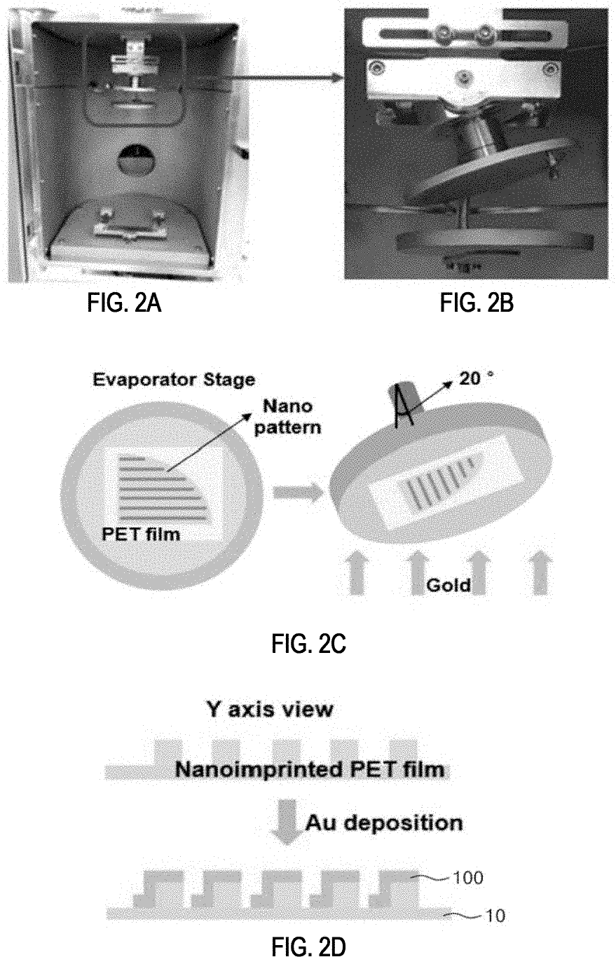

FIGS. 2A to 2D are views illustrating a printing scheme of FIG. 1, in more detail. According to an embodiment, FIGS. 2A to 2D illustrates a method for fabricating a metal nanostructure. FIG. 2A illustrates a photograph of a thermal deposition chamber, which is acquired through a digital camera FIG. 2B is an enlarged view illustrating a part marked using a rectangle of FIG. 2A FIG. 2C is a schematic view that a PET film having a nanostripe pattern is fixed to a stage film having an axis inclined at 20°. FIG. 2D illustrates a procedure of a fabricating a gold nanostructure doubly bent by depositing a PET film having a nanostripe pattern

Referring to FIGS. 2A to 2D, when the metal is deposited to the nanostructure pattern formed on the first substrate, the size and the shape of the nanostructure may be adjusted by inclining a deposition stage, such that the coated metal has a polymorphism structure. According to an embodiment, the fabrication of the nanostructure illustrated in FIGS. 2A to 2D may be performed through any one of thin film deposition processes, such as physical vapor deposition (PVD), chemical vapor deposition (CVD), and atomic layer deposition (ALD), using a thermal evaporator, an e-gun evaporator, and a sputter. Thereafter, a work of determining whether the polymorphism structure is formed through a scanning electron microscopy (SEM) may be performed.

According to an embodiment, a gold-deposited PET film having no nanopattern structure is inspected through a spectroscope to observe an localized surface plasmon resonance (LSPR) occurring in a metal polymorphism structure. According to the inspection result, the LSPR is not observed. According to an embodiment, when gold nanostructures are deposited at mutually different tilting angles of 10°, 17.5°, 20°, and 25° in a thermal evaporator, the strongest LSPR signal is measured at an angle of 20°. When the metal polymorphism structure is formed on the film having the nanopattern by the thermal evaporator, the shape of the structure may be varied depending on the angle of the stage for placing the film, thereby exhibiting various optical effects.

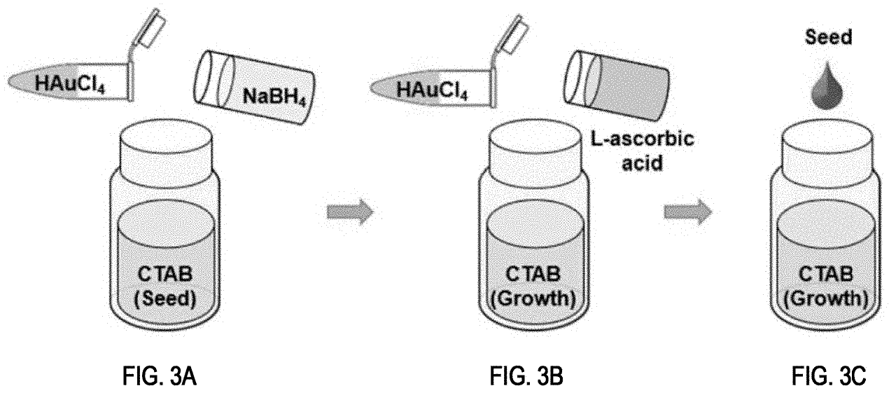

FIGS. 3A to 3C are views illustrating a method for fabricating a second nanoparticle 200, according to the inventive concept. According to an embodiment, FIGS. 3A to 3C are views illustrating a synthesis scheme of a gold nanoparticle (gold nanocube). Although FIGS. 3A to 3C and subsequent drawings illustrate that the second nanoparticle 200 and the first nanoparticle 100 are fabricated using gold, the materials for the first nanoparticle 100 and the second nanoparticle 200 are not limited to gold. For example, various metals may be employed, as long as the metals cause the plasmon phenomenon.

Referring to FIGS. 3A to 3C, when gold is prepared in the form of a nanoparticle colloidal solution, various colors and various excellent physical properties may be exhibited depending on a structure and a size. The gold nanoparticle colloidal solution may be prepared by using any one of a reduction scheme using citrate, a reduction reaction scheme using a photocatalyst, and a seed mediated growth synthesis scheme.

According to an embodiment, the seed mediated growth synthesis scheme may include most various schemes and most reliable schemes to synthesize a gold nanoparticle. According to the inventive concept, a colloidal-based nanoparticle used for a sample test may be mass-produced through the seed mediated growth synthesis scheme. According to an embodiment, the colloidal-based nanoparticle may have various forms, such as a nanocube, a nanostar, and a nanorod, and various sizes.

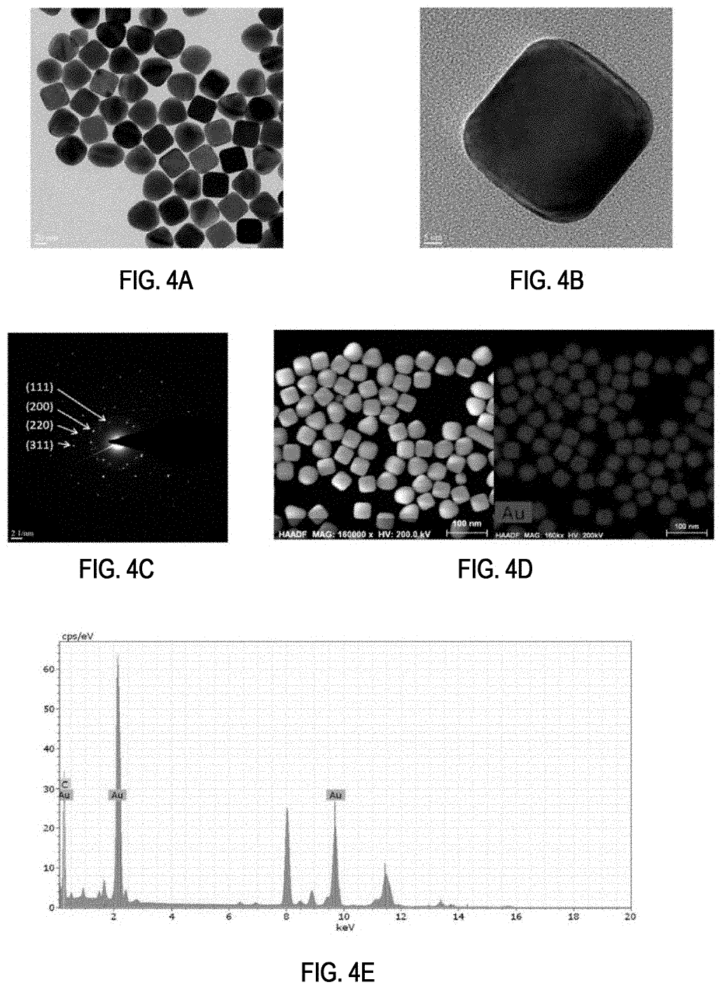

FIGS. 4A to 4E are views illustrating that the second nanoparticle 200 is prepared, according to the scheme of FIGS. 3A to 3C. According to FIGS. 4A to 4E, an observation result of a transmission electron microscopy (TEM) for the gold nanoparticle formed through the scheme of FIGS. 3A to 3C is disclosed.

Referring to FIGS. 4A to 4E, the gold nanoparticles may be recognized as having the uniform size and the uniform shape. It may be recognized through the above scheme, that the second nanoparticle having the uniform size may be formed.

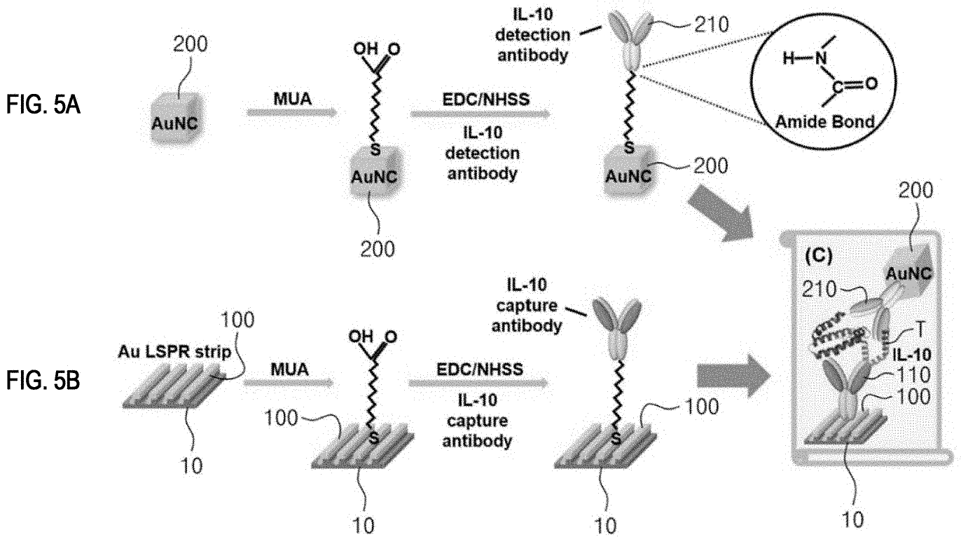

FIGS. 5A to 5C are views illustrating the diagnosis method and the plasmon phenomenon, according to the inventive concept

According to the inventive concept, the diagnosis kit employs a scheme for detecting a protein with higher sensitivity through a plasmon sensor having a plasmon property, and of utilizing the protein to determine whether a disease is present, and to determine a health condition.

The plasmon structure refers to a material, such as gold (Au), silver (Ag), and platinum (Pt), having a plasmon property due to a surface plasma oscillation. According to the inventive concept, the protein may refer to a biomarker to determine whether a disease is present, and to determine a health condition, and may be present in a normal person and a patient (human) or an animal specimen (blood, urine, saliva, or eye fluid)

Referring to FIGS. 5A to 5C, there is disclosed a plasmon sensor based on the coupling between a nanopattern formed through the first nanoparticle 100 formed on the first substrate 10, and the second nanoparticle 200. According to an embodiment, as illustrated in FIG. 5A, to realize the coupling structure of at least two plasmon structures, the surface treatment is performed with respect to the surfaces of the second nanoparticle 200 and the first nanoparticle 100. As illustrated in FIGS. 5A and 5B, after performing 11-mercaptoundecanoic acid (MUA) treatment with respect to surfaces of the second nanoparticle 200 (that is, a gold nanoparticle (nanocube) or the first nanoparticle 100 (that is, an LSPR structure), amide binding is formed through EDC/NHSS cross-linking chemistry, thereby fixing antibodies 110 and 210. In this case, antibodies 110 and 210 may be compounds. In other words, according to the inventive concept, the surface treatment may be performed with respect to the first nanoparticle 100 and the second nanoparticle 200. Then, the first nanoparticle 100 and the second nanoparticle 200 may be bonded to the compounds 110 and 210 to be bound to a target material ‘T’ through chemical bonding. Thereafter, referring to FIG. 5C, a nanostructure sandwich structure may be formed by inserting the target material ‘T’, thereby sensing the target material ‘T’. According to an embodiment, the target material ‘T’ may be a protein or cytokine.

Although claims of the inventive concept disclose that the compounds 110 and 210 are bound to the target material ‘T’, the detailed description will be made while focusing on an antigen and an antibody. However, the inventive concept is not limited to the binding between the antigen and the antibody, The binding force according to the inventive concept may be achieved through various bio-affinity binding schemes, such as binding between DNAs, binding between a DNA and an RNA, and binding between a ligand and a receptor.

According to the inventive concept, as colloidal-based nanoparticles are introduced into a chip having a plasmon property, a plasmon coupling phenomenon is derived from at least two plasmon nanostructures employing the antigen as a medium, thereby amplifying a signal.

According to an embodiment of the inventive concept, an antibody, which is specifically bound to an antigen, may be fixed to a colloidal-based nanoparticle through a scheme, such as EDC/NHSS cross-linking chemistry.

According to an embodiment, a sample may be used after pre-treated in a manner, such as centrifugation, filtering, and dilution, depending on the situation. According to the inventive concept, a changed plasmon signal may be obtained by applying an immunoassay scheme for specifically detecting a biomarker of a specific disease present on the surface of metal coated to a nanostructure having a repeated pattern

According to the inventive concept, the antibody is bound to the nanopattern and the nanoparticle through an amide binding scheme, and a sandwich structure, which is specifically bound to the biomarker, may be formed.

Conventionally, there is limitation in detection sensitivity, because only one plasmon structure is used. However, according to the inventive concept, at least two plasmon structures are used and coupled, thereby overcoming the limitation in the detection sensitivity. According to the inventive concept, the diagnosis kit may be utilized in detecting a biomaterial, such as a protein, a cytokine, a nucleic acid, a cell, an environmental pollutant, a drug, a bacterium, a virus, an E. coli, or a microorganism.

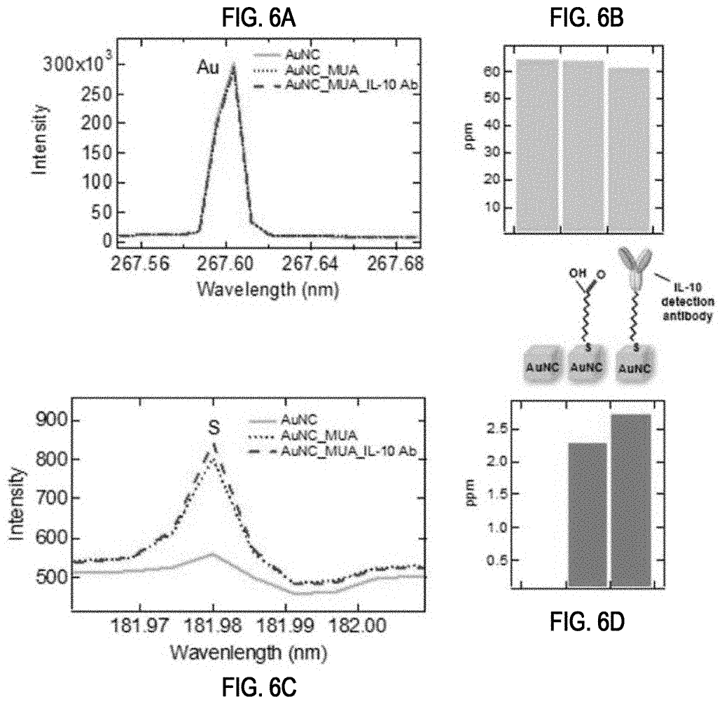

FIGS. 6A to 6D are views illustrating results obtained by analyzing results of a nanoparticle having a surface bio-functionalized with an antibody through inductively coupled plasma (ICP).

According to the inventive concept, after chemically bonding the antibody to the nanoparticle through EDC/NHSS, an atom present on the surface of the nanoparticle is analyzed to determine the bonding between the antibody and the nanoparticle. As illustrated in FIG. 6C, it may be recognized that the result after the surface treatment shows higher intensity, as compared to when the surface treatment is not performed,

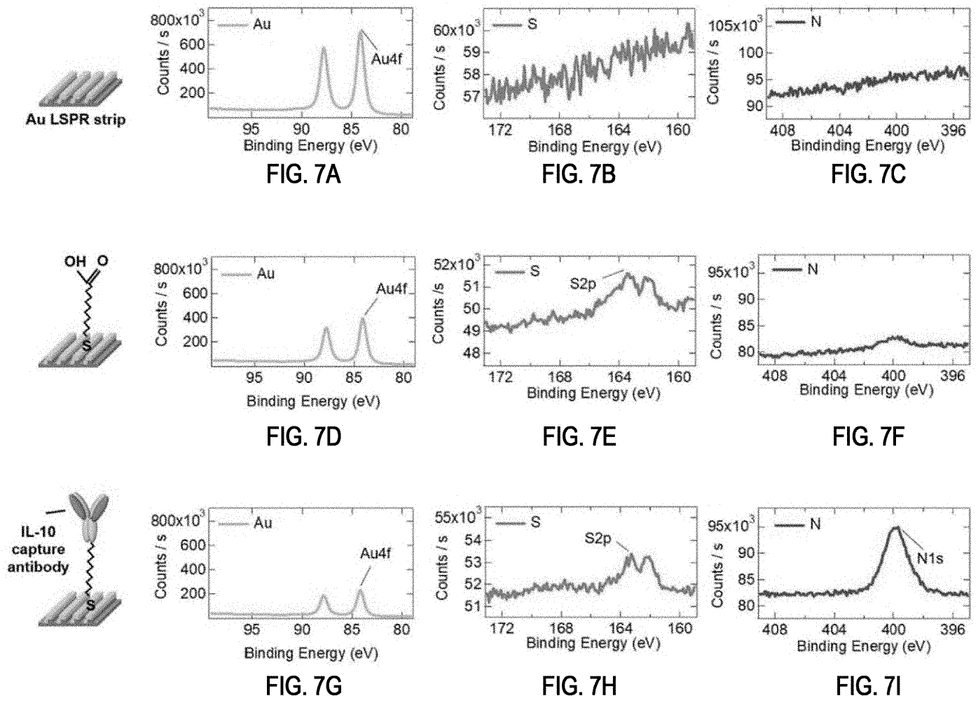

FIGS. 7A to 7I are views illustrating analysis results through a photoelectron spectroscopy, based on whether a compound is bonded to the first substrate 10 printed with the first nanoparticle 100.

In more detail, the analysis result is obtained by analyzing the nanopattern having a surface bio-functionalized with the antibody through X-ray photoelectron spectroscopy (XPS). According to the inventive concept, after chemically bonding the antibody to the nanopattern through EDC/NHSS, an atom present on the surface of the nanoparticle is analyzed to determine the bonding between the antibody and the nanopattern. Referring to FIGS. 7A to 7I, it may be recognized that bonding energy is remarkably increased in the first nanoparticle 100 bonded to the compound.

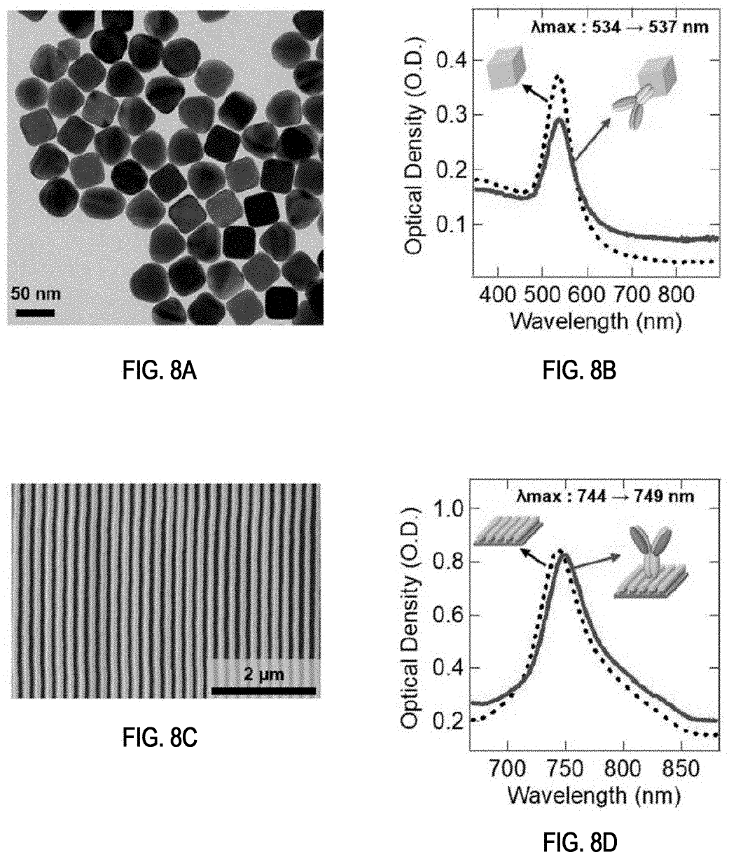

FIG. 8A to 8D illustrates results determined through an absorption spectroscope after an antibody is fixed to the nanopattern formed using the second nanoparticle 200 and the first nanoparticle 100. After bonding the antibody to the nanoparticles and the nanopattern, the change in a plasmon phenomenon is measured through the absorption spectroscope, as well as the ICP or the XPS, thereby determining whether the antibody is boded to the second nanoparticle 200 and the first nanoparticle 100. When the antibody is bonded to the surface of the nanoparticle or the nanopattern, an extinction peak is shifted due to a surface plasmon phenomenon. In FIG. 8B, a black dotted line and a red bold line indicate results obtained by observing a second nanoparticle before an antibody is fixed to the second nanoparticle, and a second nanoparticle after the antibody is fixed to the second nanoparticle, through a spectroscope It may be recognized that the highest peak value is shifted from 534 nm to 537 nm, thereby identifying that the antibody is bonded to the surface of the nanopattern or the nanoparticle. In FIG. 8D, a black dotted line and a red bold line indicate results obtained by observing a first nanoparticle before an antibody is fixed to the first nanoparticle, and a first nanoparticle after the antibody is fixed to the first nanoparticle, through a spectroscope It may be recognized that the highest peak value is shifted from 744 nm to 749 nm, thereby identifying that the antibody is bonded to the surface of the nanopattern or the nanoparticle.

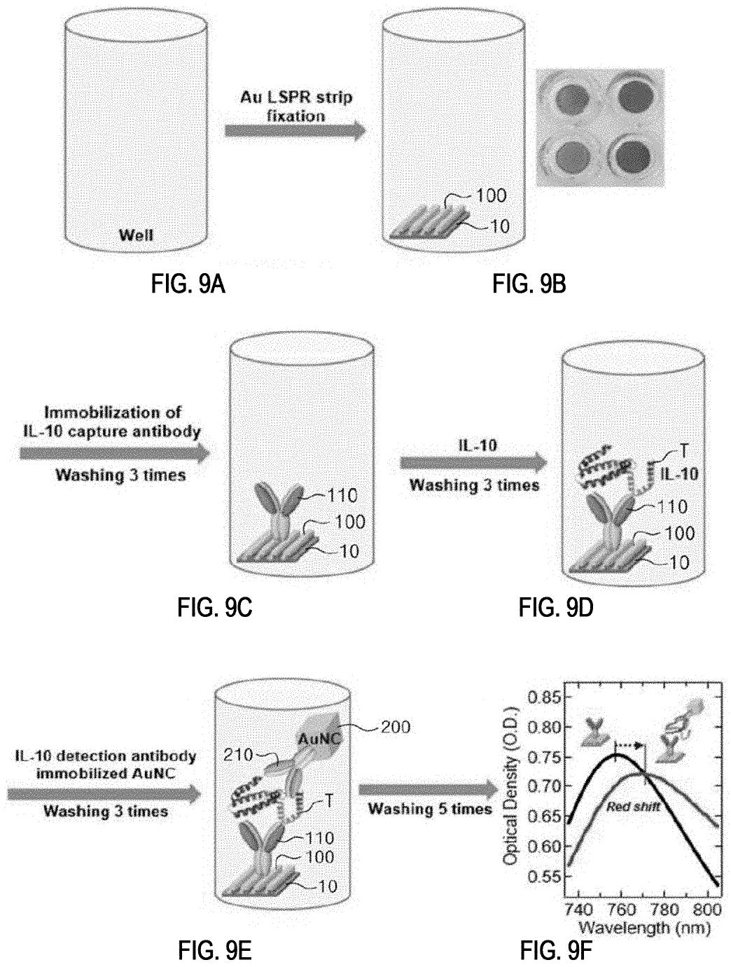

FIGS. 9A to 9F are views illustrating a method for manufacturing a diagnosis kit and a diagnosis method, according to an embodiment of the inventive concept.

Referring to FIGS. 9A to 9F, there is disclosed a method for manufacturing a diagnosis kit, according to the inventive concept. Referring to FIG. 9A, there is disclosed a plate. According to an embodiment, the plate may be a microplate. According to an embodiment, the plate may have a 6/12/24/96/384 microwell structure. According to an embodiment, the first substrate 10, which is nanoprinted with the first nanoparticle 100, may be placed in the plate. Referring to FIG. 9C, the antibody 110 may be coated on the first nanoparticle 100. Thereafter, the target material ‘T’ may be injected. According to an embodiment, the target material ‘T’ may be a protein. Thereafter, the metal nanoparticle coated with the antibody 210, that is, the second nanoparticle 200 may be injected. Subsequently, the wavelength change of an extinction peak may be observed by measuring absorbance. According to the inventive concept, the concentration of the biomarker may be recognized, based on that the extinction peak is shifted due to the coupling phenomenon between the nanostripe and the nanoparticle.

Referring to FIGS. 9A to 9F, the plasmon sensor may be manufactured by using the diagnosis kit, according to the inventive concept. According to the inventive concept, the plasmon sensor has flexible and transparent properties. Accordingly, the plasmon sensor may be mounted on a microwell plate (having a 6/12/24/96/384 structure) and may serve as a microplate reader to have an analyzing function. According to the inventive concept, the plasmon sensor may be applied to a 96 well plate such that analysis may be performed in bench top equipment, such as a microplate reader, However, the inventive concept is not limited to the embodiment, but various plates may be employed.

According to the inventive concept, a kit for diagnosing a disease may be configured based on a nanostripe-based plasmon material platform and a nanoparticle-based sample test reagent. According to an embodiment, the kit for diagnosing the disease may include an antibody-coated LSPR nanostructure (a first substrate bonded to a compound and coated with a first nanoparticle), a microplate, an antibody-coated gold nanoparticle reagent (a second nanoparticle bonded to a compound), a standard solution, and a washing buffer.

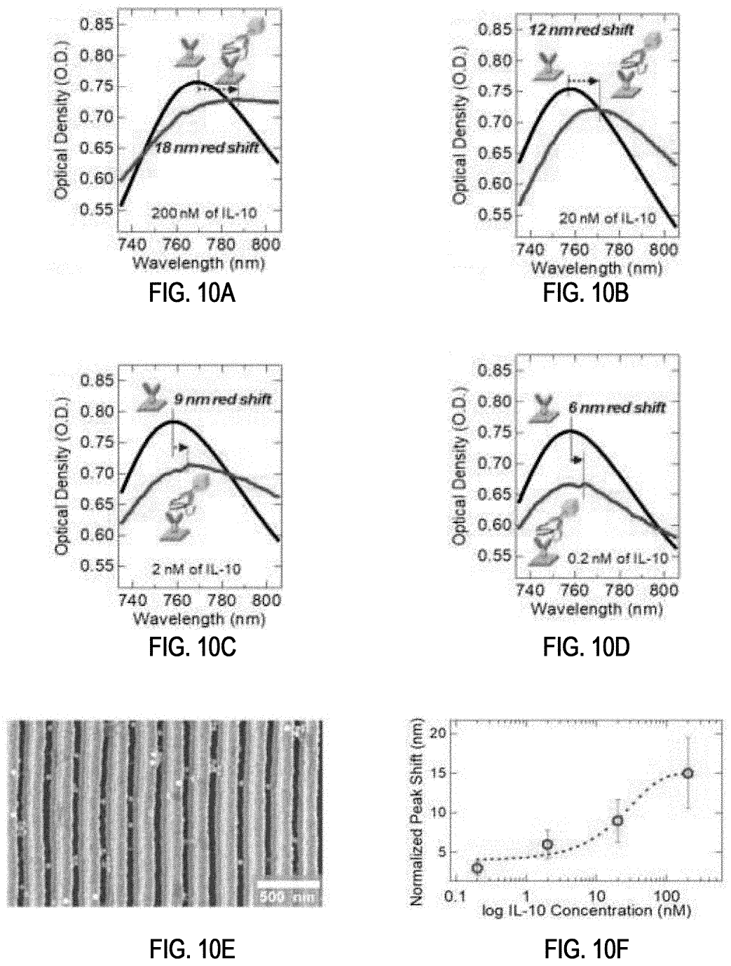

FIGS. 10A to 10F are views illustrating that a target material ‘T’ is detected, according to an embodiment. FIGS. 10A to 10F show results obtained by determining interleukin-10 detection performance through the absorption spectroscope in a plasmon sensor based on nanoparticle-nanopattern coupling. FIGS. 10A to 10F show the results obtained by measuring the concentration of the target material ‘T’, based on that the extinction peak is shifted due to the nanoparticle-nanopattern coupling. FIGS. 10A to 10D are graphs illustrating the extinction peak is shifted, as the concentration of the target material ‘T’ is shifted from 200 nanomole to 20, 2, and 0.2 nanomole. A black bold line indicates a result obtained by observing a sandwich structure of an antibody-interleukin-10-antibody through a spectroscope using the first nanoparticle to which an antibody is fixed. A red bold line indicates a result obtained by observing a sandwich structure of an antibody-interleukin-10-antibody through a spectroscope using the second nanoparticle to which an antibody is fixed. The extinction peak is determined as being shifted to 18/12/9/6 nm at each concentration.

FIG. 10E is a graph illustrating the shifted extinction peak depending on the change in concentration. FIG. 10F illustrates a result obtained by observing the nanopattern-nanoparticle coupling-based plasmon sensor having the sandwich structure, after obtaining the graph of FIG. 10A with respect to 200 nanomole of interleukin-10.

As the extinction peak is determined as being shifted through the nanoparticle-nanopattern plasmon coupling as described above, a target material ‘T’ in a nanomole level may be detected.

According to an embodiment, the interleukin-10 protein may be detected in the nanomole level. In addition, a chip, in which an immunodiagnostic scheme is applied to a plasmon sensor, may be observed through a scanning electron microscope (SEM).

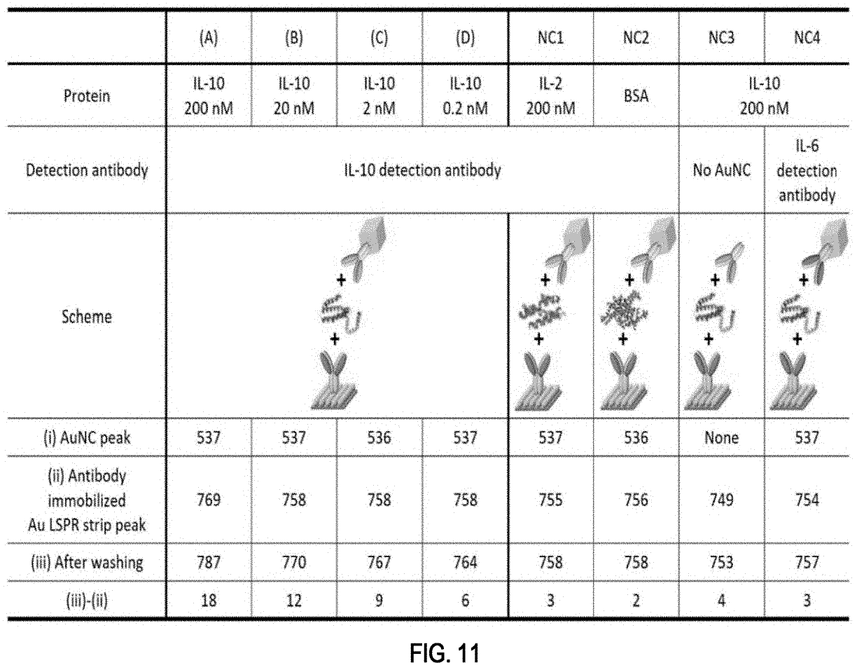

FIG. 11 illustrates a table showing the comparison between control signals in a diagnosis kit, according to an embodiment of the inventive concept.

In more detail, FIG. 11 illustrates the comparison result between a signal for positive control and a signal for negative control (NC) in a nanoparticle-nanopattern coupling-based plasmon sensor. As illustrated in FIG. 11, items NC1 and NC2 correspond to the size of a signal measured, based on non-specific binding using interleukin-2 and bovine serum album (BSA), instead of interleukin-10, item NC3 corresponds to that a nanoparticle is not used, and item NC4 corresponds to that an antibody, which is specifically bound to interleukin-6, is fixed to a nanoparticle, instead of an antibody employing interleukin-10 as a target. It may be determined that the extinction peak shifted is hardly observed in the case of the negative control NC. To the contrary, it may be determined that the extinction peak shifted is observed in the case of the positive control, which is caused by a signal from the target material ‘T’. Accordingly, the excellent performance of the nanoparticle-nanopattern coupling-based plasmon sensor may be determined.

While the inventive concept has been described with reference to embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A method for diagnosing a target material by using a first substrate printed with a first nanoparticle, the method comprising:
positioning a second nanoparticle, which is bonded to a compound to be bound to the target material, at a distance adjacent to the first substrate.

2. The method of claim 1, wherein a compound to be bound to the target material is bonded to the first nanoparticle.

3. The method of claim 2, further comprising:
applying the target material.

4. The method of claim 3, further comprising:
detecting the target material through a plasmon phenomenon of the first nanoparticle and the second nanoparticle.

5. The method of claim 1, wherein a shape of the second nanoparticle is one of a cube, a rectangular parallelepiped, a sphere, and a cylinder.

6. The method of claim 1, wherein the first nanoparticle and the second nanoparticle is one of metals causing a plasmon phenomenon.

7. The method of claim 1, wherein the adjacent distance is in a range of 30 nm to 200 nm.

8. A diagnostic kit for diagnosing a target material, the diagnostic kit comprising:
a first substrate printed with a first nanoparticle; and
a second nanoparticle to be coupled to the first nanoparticle.

9. The diagnostic kit of claim 8, wherein a compound to be bound to the target material is bonded to the first nanoparticle and the second nanoparticle.

10. The diagnostic kit of claim 9, wherein the second nanoparticle is provided at a distance adjacent to the first nanoparticle.

11. The diagnostic kit of claim 8, wherein a shape of the second nanoparticle is one of a cube, a rectangular parallelepiped, a sphere, and a cylinder.

12. The diagnostic kit of claim 8, wherein the first nanoparticle and the second nanoparticle is one of metals causing a plasmon phenomenon.

13. The diagnostic kit of claim 10, wherein the adjacent distance is in a range of 30 nm to 200 nm.

14. A method for manufacturing a diagnosis kit to diagnose a target material, the method comprising:
printing a first nanoparticle on a first substrate; and
attaching a diagnosis kit printed with the first nanoparticle into a plate.

15. The method of claim 14, further comprising:
bonding a compound to be bound to the target material to the first nanoparticle.

16. The method of claim 15, further comprising:
positioning a second nanoparticle, which is bonded to the compound to be bound to the target material, into the plate.

17. The method of claim 16, wherein a distance between the first nanoparticle and the second nanoparticle is in a range of 30 nm to 200 nm.
